(19) United States
(12) Patent Application Publication
(10) Pub. No.: US 2024/0417360 A1
(43) Pub. Date: Dec. 19, 2024

(54) DEHYDRATION OF LACTIC ACID IN THE LIQUID PHASE

(71) Applicant: LAKRIL TECHNOLOGIES CORP., Chicago, IL (US)

(72) Inventor: Christopher P. Nicholas, Evanston, IL (US)

(21) Appl. No.: 18/739,936

(22) Filed: Jun. 11, 2024

Related U.S. Application Data
(60) Provisional application No. 63/521,029, filed on Jun. 14, 2023.

(51) Int. Cl. C07C 51/377; B01J 29/08; B01J 29/60; C07C 67/327

(52) U.S. Cl. CPC C07C 51/377; B01J 29/08; B01J 29/60; C07C 67/327

(57) ABSTRACT

The catalytic dehydration of lactic acid to acrylic acid has traditionally occurred in the gas phase. The disclosure provides a process occurring in the liquid phase over a catalyst. The process includes pressuring a feedstream, contacting the feedstream with a catalyst, converting the feedstream into a product stream, and recovering acrylic acid, alkyl acrylate, or a cation-balanced acrylate from the product stream.

This patent application claims priority to and benefit of U.S. Provisional Application No. 63/521,029, filed Jun. 14, 2023, which is incorporated herein by reference.

### FIELD

The field is the catalytic dehydration of lactic acid to acrylic acid, occurring in the liquid phase.

### BACKGROUND

Acrylic acid and its alkyl acrylate derivatives are high volume chemicals at the base of the paints, coatings, adhesives, and superabsorbent polymer industries. Acrylics are currently produced from oxidation of petroleum-derived propylene by two step oxidation via acrolein over bismuth molybdate catalysts. A bio-based, sustainable route to these high volume chemicals would be desirable, provided cost for such a process is economically competitive. One path to bio-based, sustainable acrylic acid is the dehydration of lactic acid or lactate compounds.

The dehydration of lactic acid has been studied in the gas phase over the last 15 years directed towards finding catalyst formulations and/or process innovations to achieve commercialization routes to bio-acrylics without success. Both dehydration catalysts and integrated processes for lactic-to-acrylic and improved fermentation yields and integrated processes for 3-hydroxypropanoic acid (3HP) to acrylic have been studied, but yields of acrylics are usually less than 75-80% and have low space time yield. An improved catalyst comprises a solid acid and a multifunctional flexible modifier comprising a functional group such as an amine which has shown 92% yield. This yield was obtained at 0.76 volume percent methyl lactate in the N2 feed, leading to a low space-time yield.

### BRIEF SUMMARY

By operating the dehydration of lactic acid and/or lactate compounds at elevated pressure in the liquid phase, catalyst lifetime and increased space-time yield occur.

In examples, a process for catalytic dehydration of lactic acid, an alkyl lactate, or a cation-balanced lactate, comprises: pressurizing a feedstream comprising lactic acid, an alkyl lactate or a cation-balanced lactate to a pressure greater than 10 barg and sufficient to have a liquid phase; contacting the feedstream with a catalyst; converting the feedstream into a product stream comprising acrylic acid, alkyl acrylate or a cation-balanced acrylate over the catalyst; and recovering the acrylic acid, alkyl acrylate or a cation-balanced acrylate from the product stream. The process may further comprise decompressing the product stream prior to recovering the acrylic acid, alkyl acrylate, or a cation-balanced acrylate. The process may additionally or alternatively comprise contacting the catalyst with a second feedstream comprising an alcohol. The contacting may be carried out at a temperature of from about 200° C. to 325° C. The catalyst may comprise a FAU zeolite. The catalyst may comprise a LTL zeolite. In examples, the pressure may be greater than 25 barg. Selectivity to acrylic acid during dehydration may be greater than 50 mol %. The feedstream may comprise less than 50 wt % water. The alcohol of the second feedstream may comprise methanol, ethanol, butanol, or mixtures thereof.

In examples, a process for catalytic dehydration of a lactic source comprises: pressurizing a feedstream comprising a lactic source to a pressure greater than 10 barg and sufficient to have a liquid phase; contacting the feedstream with a catalyst; converting the feedstream into a product stream comprising acrylic acid, alkyl acrylate or a cation-balanced acrylate over the catalyst; and recovering the acrylic acid, alkyl acrylate or a cation-balanced lactate, or combination thereof. The process may further comprise contacting the catalyst with a second feedstream comprising an alcohol.

Again, The contacting may be carried out at a temperature of from about 200° C. to 325° C. The catalyst may comprise a FAU zeolite. The catalyst may comprise a LTL zeolite. In examples, the pressure may be greater than 25 barg. Selectivity to acrylic acid during dehydration may be greater than 50 mol %. The feedstream may comprise less than 50 wt % water. The alkyl lactate may comprise methyl lactate, ethyl lactate, butyl lactate, or combinations thereof. The alcohol of the second feedstream may comprise methanol, ethanol, butanol, or mixtures thereof.

In examples, a process for catalytic dehydration of a lactic source comprises: pressurizing a feedstream comprising a lactic source to a pressure greater than 25 barg and sufficient to have a liquid phase; contacting the feedstream with a catalyst comprising a FAU zeolite; converting the feedstream into a product stream comprising acrylic acid, alkyl acrylate or a cation-balanced acrylate over the catalyst; and recovering the acrylic acid, alkyl acrylate or a cation-balanced acrylate from the product stream; wherein the lactic source comprises lactic acid, an alkyl lactate, a cation-balanced lactate, or a combination thereof.

### Definitions

As used herein, the term “predominant” or “predominate” means greater than 50%, suitably greater than 75% and preferably greater than 90%.

As used herein, the term “substantially” can mean an amount of at least generally about 70%, preferably about 80%, and optimally about 90%, by weight, of a compound or class of compounds in a stream.

As used herein, the term “stream” can include various hydrocarbon molecules and other substances. Moreover, the term “stream comprising Cₓ hydrocarbons” or “stream comprising Cₓ olefins” can include a stream comprising hydrocarbon or olefin molecules, respectively, with “x” number of carbon atoms, suitably a stream with a majority of hydrocarbons or olefins, respectively, with “x” number of carbon atoms and preferably a stream with at least 75 wt % hydrocarbons or olefin molecules, respectively, with “x” number of carbon atoms. Moreover, the term “stream comprising Cₓ+ hydrocarbons” or “stream comprising Cₓ+ olefins” can include a stream comprising a majority of hydrocarbon or olefin molecules, respectively, with more than or equal to “x” carbon atoms and suitably less than 10 wt % and preferably less than 1 wt % hydrocarbon or olefin molecules, respectively, with x−1 carbon atoms. Lastly, the term “Cₓ-stream” can include a stream comprising a majority of hydrocarbon or olefin molecules, respectively, with less than or equal to “x” carbon atoms and suitably less than 10 wt % and preferably less than 1 wt % hydrocarbon or olefin molecules, respectively, with x+1 carbon atoms.

As used herein, the term “zone” can refer to an area including one or more equipment items and/or one or more subzones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “vapor” can mean a gas or a dispersion that may include or consist of one or more hydrocarbons.

As used herein, the term “overhead stream” can mean a stream withdrawn at or near a top of a vessel, such as a column.

As used herein, the term “bottom stream” can mean a stream withdrawn at or near a bottom of a vessel, such as a column.

As depicted, process flow lines in the figures can be referred to interchangeably as, for example, lines, pipes, feeds, gases, products, discharges, parts, portions, or streams.

As used herein, “bypassing” with respect to a vessel or zone means that a stream does not pass through the zone or vessel bypassed although it may pass through a vessel or zone that is not designated as bypassed.

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottom stream back to the bottom of the column. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column.

As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, “taking a stream from” means that some or all of the original stream is taken.

As used herein, “space-time yield” or “STY” means the product yield per unit volume of catalyst and per unit time.

As used herein, “lactic” or “lactic source” indicates lactic acid, a lactate derivative thereof, or combinations thereof. More properly, lactic acid is 2-hydroxypropanoic acid and comprises two enantiomers, the R and S. Ethyl lactate is one example of an alkyl lactate derivative and is the ethyl ester of lactic acid. Potassium lactate is an example of a cation-balanced lactate.

As used herein, “acrylic” or “acrylic product” indicates acrylic acid, an acrylate derivative thereof, or combinations thereof. More properly, acrylic acid is propenoic acid and is achiral. Ethyl acrylate is one example of an alkyl acrylate derivative and is the ethyl ester of acrylic acid. Potassium acrylate is an example of a cation-balanced acrylate.

### DETAILED DESCRIPTION

The disclosure provides a process for liquid phase dehydration of a lactic source comprising lactic acid, an alkyl lactate, a cation-balanced lactate or a combination thereof. Lactic acid may undergo acid-catalyzed heterolytic decarbonylation, homolytic decarboxylation, and/or heterolytic dehydration. Dehydration leads to acrylic acid formation. Acrylic acid and its alkyl acrylate derivatives are desired to form polymers used within the paints, coatings, adhesives, and superabsorbent polymer industries. High space-time yields of bio-based acrylic acid are desired to lower cost of production.

The process for dehydration may comprise pressurizing a feedstream. The feedstream stream may comprise lactic acid, an alkyl lactate, a cation-balanced lactate or combinations thereof.

Lactic acid or derivatives thereof may be obtained from fermentation of sugar sources. Lactic acid production methods by fermentation are often preferred over chemical synthesis alternatives that produce a mixture of both R and S isomers. The product of microbial fermentation depends on the organism used. Microbial fermentation can result in a mixture of two isomers or in a stereospecific form of optically pure lactic acid. The desired stereospecificity of the product depends on the intended use. Chirality of the lactic acid may not be important for dehydration.

The lactic source may comprise lactic acid, a lactate balanced by a Group I cation or ammonium, a lactate balanced by a Group II cation, an alkyl lactate, or a combination thereof. The Group I cation may comprise lithium, sodium, potassium, cesium, or combinations thereof. The Group II cation may comprise magnesium, calcium, strontium, barium, or combinations thereof. The alkyl group may comprise a C₁-C₄ alkyl group such as but not limited to methyl, ethyl, isopropyl, and butyl. The alkyl lactate may comprise methyl lactate, ethyl lactate, butyl lactate, or combinations thereof.

The feedstream may comprise water. The feedstream may comprise less than 50 wt % water, or less than 25 wt % water or less than 10 wt % water.

The feedstream may be pressurized to a pressure greater than about 10 barg. The minimum pressure required may be determined by the feed composition and reaction conditions used during reaction. Pressurization may occur to a pressure greater than about 25 barg. The pressure may be less than about 350 barg or less than about 300 barg or less than about 250 barg.

Dehydrating the reactant includes contacting the feedstream with a catalyst. The catalyst may be a solid acid catalyst, which may include surfaces defining pores and a multiplicity of acid sites on the surfaces. The acid sites may be Bronsted acid sites or ion-exchanged acid sites. In one embodiment, a multifunctional component may be coupled to the surfaces of the solid acid catalyst. Each multifunctional component may include at least two functional groups, and each functional group may be configured to accept a proton from an acid site of the multiplicity of acid sites. Such functional groups include but are not limited to amine, imine, alcohol, ether, halide, alkyl halide, thiol, and sulfide groups. The reactant may be dehydrated to yield a product comprising acrylic acid, alkyl acrylate or a cation-balanced acrylate, or a combination thereof.

The catalyst may comprise a zeolite. Zeolites are crystalline aluminosilicate compositions which are microporous and which are formed from corner sharing AlO₂ and SiO₂ tetrahedra. Numerous zeolites, both naturally occurring and synthetically prepared, are used in various industrial processes. Synthetic zeolites are prepared via hydrothermal synthesis employing suitable sources of Si, Al and structure directing agents such as alkali metals, alkaline earth metals, amines, or organoammonium cations. The structure directing agents reside in the pores of the zeolite and are believed to be largely responsible for the particular structure that is ultimately formed. These species balance the framework charge associated with aluminum and can also serve as space fillers. Zeolites are characterized by having pore openings of uniform dimensions, having a significant ion exchange capacity, and being capable of reversibly desorbing an adsorbed phase which is dispersed throughout the internal voids of the crystal without significantly displacing any atoms which make up the permanent zeolite crystal structure. Zeolites can be used as catalysts for hydrocarbon conversion reactions, which can take place on outside surfaces as well as on internal surfaces within the pore.

Over 250 different zeolite topologies are recognized today. As used herein, zeolites may be referred to by proper name, such as Zeolite Y or LZ-210, or by structure type code, such as FAU. These three-letter codes indicate atomic connectivity and hence pore size, shape, and connectivity for the various known zeolites. The list of these codes may be found in the Atlas of Zeolite Framework Types, which is maintained by the International Zeolite Association Structure Commission at http://iza-structure.org/databases/. One such topology is FAU, often referred to as faujasite after a natural mineral composition of the topology.

Channel systems for known zeolites are described in the Atlas of Zeolite Framework Types as having zero-dimensional, one-dimensional, two-dimensional or three-dimensional pore systems. A zero-dimensional pore system has no pore system running through the zeolite crystal, instead only possessing internal cages. A one-dimensional pore system contains a pore delimited by 8-membered rings or larger that run substantially down a single axis of a crystal. Two-dimensional pore (channel) containing zeolites contain intersecting pores that extend through two-dimensions of a zeolite crystal, but travel from one side of the third dimension of the zeolite crystal to the other side of the third dimension is not possible, while zeolites containing three-dimensional channel systems have a system of pores intersecting, often in a mutually orthogonal manner, such that travel from any side of a zeolite crystal to another is possible. FAU is a three-dimensional zeolite comprising cages and pore openings delimited by 12-membered rings.

The catalyst may comprise a FAU zeolite. In another embodiment, the catalyst may comprise a LTL zeolite. The catalyst may additionally comprise potassium.

The process may further comprise contacting the catalyst with a second feedstream comprising an alcohol. The second feedstream may be combined with the first feedstream into a combined feedstream, or may be fed separately. Thus, the feedstream may comprise a lactic source and an alcohol. The alcohol may comprise methanol, ethanol, butanol, or mixtures thereof.

The feed stream may be converted into a product stream over the catalyst. The product stream may comprise acrylic acid, alkyl acrylate, a cation-balanced acrylate, or combinations thereof. The product stream may comprise acrylic acid, an acrylate balanced by a Group I cation or ammonium, an acrylate balanced by a Group II cation, an alkyl acrylate, or a combination thereof. The Group I cation may comprise lithium, sodium, potassium, cesium, or combinations thereof. The Group II cation may comprise magnesium, calcium, strontium, barium, or combinations thereof. The alkyl group may comprise a C₁-C₄ alkyl group such as but not limited to methyl, ethyl, isopropyl, and butyl. The alkyl acrylate may comprise methyl acrylate, ethyl acrylate, butyl acrylate, or combinations thereof.

Contacting of the feedstream with the catalyst to form a product stream may occur at a temperature of from about 200° C. or from about 250° C. to about 300° C. or to about 325° C. or to about 350° C. and less than about 375° C. These temperatures reflect temperatures that are high enough to obtain conversion of the lactic source, but low enough that side reactions don't become the dominant pathway. Conversion of the lactic source during reaction may be greater than about 50% or greater than about 75% or greater than about 95% on a molar basis. Selectivity of the lactic source to dehydration products may be greater than about 50 mol %, or greater than about 75 mol % or greater than about 85 mol %. Selectivity of the lactic source to acrylic products may be greater than about 50 mol %, or greater than about 75 mol % or greater than about 85 mol %.

A further step in the process may comprise recovering the acrylic acid, alkyl acrylate, cation-balanced acrylate, or combinations thereof from the product stream. Recovering acrylic product may comprise liquid-liquid separation, distillation, membrane contactors, other known separation techniques, and combinations thereof. The product stream may be depressurized to a pressure lower than the reaction pressure prior to recovering the acrylic product. The product stream may be depressurized to a pressure less than about 100 barg or less than about 10 barg prior to recovering the acrylic product.

### Example 1

The following is one example that may be undertaken for the process described herein. Pressurizing a feedstream comprising 10 wt % lactic acid in water to 40 barg. Feeding the feedstream to a reactor containing a catalyst comprising FAU zeolite which is at a reaction temperature of 300° C. Observing dehydration of lactic acid to acrylic acid using HPLC. Measuring selectivity by dividing moles of acrylic acid observed by moles of other products.

### Example 2

The following is another example that may be undertaken for the process described herein. Pressurizing a feedstream comprising 80 wt % methyl lactate in 20% methanol to 75 barg. Feeding the feedstream to a reactor containing a catalyst comprising FAU zeolite which is at a reaction temperature of 275° C. Observing dehydration of methyl lactate to acrylic acid and methyl acrylate using HPLC. Measuring selectivity by dividing moles of acrylics observed by moles of other products.

### Example 3

In another example, 200 mg of Na-FAU zeolite (CBV-100) was loaded in a continuous flow reactor. The reactor comprised ¼″ stainless steel tubing with the catalyst loaded between layers of glass wool. The reactor was heated to reaction temperature. Pre-heat on feed line was 250° C. A liquid feed of 4 Molar lactic acid in water was introduced using an HPLC gear pump, with pressure controlled by a back pressure regulator after product collection set to the desired pressure. Flow rate was 0.1 mL/min which allowed a 600 s resonance time. Reactor temperature was 350° C. and pressure was 200 barg. Reaction products were condensed into an ice/water cooled bottle. Yield of acrylic acid from lactic acid was greater than 30%. This is a lower yield bound. The upper bound is uncertain due to incomplete capture of all gas phase products. Lactic acid, acrylic acid, and two additional peaks were observed and believed to be di-acrylic acid and tri-acrylic acid using offline HPLC analysis on a Rezex ROA-H+ column and H₂SO₄ eluent phase. Selectivity was measured by dividing moles of acrylics observed by moles of other products and yield by multiplying by lactic acid fed.

### Example 4

In another example, 200 mg of Na-FAU zeolite (CBV-100) was loaded in a continuous flow reactor. The reactor comprised ¼″ stainless steel tubing with the catalyst loaded between layers of glass wool. The reactor was heated to reaction temperature. Pre-heat on feed line was 200° C. A liquid feed of 0.4 Molar lactic acid in water was introduced using an HPLC gear pump, with pressure controlled by a back pressure regulator after product collection set to the desired pressure. Flow rate was 1 mL/min which allowed a 60 s resonance time. Reactor temperature was 200° C. and pressure was 50 barg. Reaction products were condensed into an ice/water cooled bottle. Yield of acrylic acid from lactic acid was low. Mostly lactic acid, and some acrylic acid were observed using HPLC. Selectivity was measured by dividing moles of acrylics observed by moles of other products and yield by multiplying by lactic acid fed.

### Example 5

In another example, 200 mg of Na-FAU zeolite (CBV-100) was loaded in a continuous flow reactor. The reactor comprised ¼″ stainless steel tubing with the catalyst loaded between layers of glass wool. The reactor was heated to reaction temperature. Pre-heat on feed line was 250° C. A liquid feed of 4 Molar lactic acid in water was introduced using an HPLC gear pump, with pressure controlled by a back pressure regulator after product collection set to the desired pressure. Flow rate was 1 mL/min which allowed a 60 s resonance time. Reactor temperature was 350° C. and pressure was 200 barg. Reaction products were condensed into an ice/water cooled bottle. Yield of acrylic acid from lactic acid was greater than 20%. This is a lower yield bound. The upper bound is uncertain due to incomplete capture of all gas phase products. Lactic acid, acrylic acid, and two additional peaks were observed and believed to be di-acrylic acid and a small amount of tri-acrylic acid using HPLC. Selectivity was measured by dividing moles of acrylics observed by moles of other products and yield by multiplying by lactic acid fed.

### Specific Embodiments

While the following is described in conjunction with specific embodiments, it will be understood that this description is intended to illustrate and not limit the scope of the preceding description and the appended claims.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

In one embodiment, a process for catalytic dehydration of lactic acid, an alkyl lactate, or a cation-balanced lactate is disclosed, the process comprising: Pressurizing a feedstream comprising lactic acid, an alkyl lactate or a cation-balanced lactate to a pressure greater than 10 barg and sufficient to have a liquid phase; contacting the feedstream with a catalyst; converting the feedstream into a product stream comprising acrylic acid, alkyl acrylate or a cation-balanced acrylate over the catalyst; and recovering the acrylic acid, alkyl acrylate or a cation-balanced acrylate from the product stream.

In a further embodiment, the process may further comprise decompressing the product stream prior to recovering the acrylic acid, alkyl acrylate, or a cation-balanced acrylate.

In a further embodiment, the contacting may be carried out at a temperature of from about 200° C. to 325° C.

In a further embodiment, the catalyst may comprise a FAU zeolite.

In a further embodiment, the catalyst may comprise a LTL zeolite.

In a further embodiment, the pressure is greater than 25 barg.

In a further embodiment, a selectivity to acrylic acid during dehydration is greater than 50 mol %.

In a further embodiment, the processes may further comprise contacting the catalyst with a second feedstream comprising an alcohol.

In a further embodiment, the feedstream may comprise less than 50 wt % water.

In a further embodiment, a process for catalytic dehydration of a lactic source, the process is disclosed, the process comprising: pressurizing a feedstream comprising a lactic source to a pressure greater than 10 barg and sufficient to have a liquid phase;
contacting the feedstream with a catalyst; converting the feedstream into a product stream comprising acrylic acid, alkyl acrylate or a cation-balanced acrylate over the catalyst;
   and recovering the acrylic acid, alkyl acrylate or a cation-balanced acrylate from the product stream; where the lactic source comprises lactic acid, an alkyl lactate, a cation-balanced lactate, or combinations thereof.

In a further embodiment, the contacting is carried out at a temperature of from about 200° C. to 325° C.

In a further embodiment, the catalyst comprises a FAU zeolite.

In a further embodiment, the catalyst comprises a LTL zeolite.

In a further embodiment, the pressure is greater than 25 barg.

In a further embodiment, a selectivity to acrylic acid during dehydration is greater than 50 mol %.

In a further embodiment, the processes include contacting the catalyst with a second feedstream comprising an alcohol.

In a further embodiment, the alcohol comprises methanol, ethanol, butanol, or mixtures thereof.

In a further embodiment, the alkyl lactate comprises methyl lactate, ethyl lactate, butyl lactate, or combinations thereof.

In a further embodiment, the feedstream comprises less than 50 wt % water.

A process for catalytic dehydration of a lactic source is disclosed, the process comprising: pressurizing a feedstream comprising a lactic source to a pressure greater than 25 barg and sufficient to have a liquid phase; contacting the feedstream with a catalyst comprising a FAU zeolite; converting the feedstream into a product stream comprising acrylic acid, alkyl acrylate or a cation-balanced acrylate over the catalyst;
and recovering the acrylic acid, alkyl acrylate or a cation-balanced acrylate from the product stream; where the lactic source comprises lactic acid, an alkyl lactate, a cation-balanced lactate, or combinations thereof.

1. A process for catalytic dehydration of lactic acid, an alkyl lactate, or a cation-balanced lactate, the process comprising:
Pressurizing a feedstream comprising lactic acid, an alkyl lactate or a cation-balanced lactate to a pressure greater than 10 barg and sufficient to have a liquid phase;
contacting the feedstream with a catalyst;
converting the feedstream into a product stream comprising acrylic acid, alkyl acrylate or a cation-balanced acrylate over the catalyst;
and recovering the acrylic acid, alkyl acrylate or a cation-balanced acrylate from the product stream.

2. The process of claim 1 further comprising decompressing the product stream prior to recovering the acrylic acid, alkyl acrylate, or a cation-balanced acrylate.

3. The process of claim 1 wherein the contacting is carried out at a temperature of from about 200° C. to 325° C.

4. The process of claim 1 wherein the catalyst comprises a FAU zeolite.

5. The process of claim 1 wherein the catalyst comprises a LTL zeolite.

6. The process of claim 1 wherein the pressure is greater than 25 barg.

7. The process of claim 1 wherein a selectivity to acrylic acid during dehydration is greater than 50 mol %.

8. The process of claim 7 further comprising contacting the catalyst with a second feedstream comprising an alcohol.

9. The process of claim 1 wherein the feedstream comprises less than 50 wt % water.

10. A process for catalytic dehydration of a lactic source, the process comprising:
pressurizing a feedstream comprising a lactic source to a pressure greater than 10 barg and sufficient to have a liquid phase;
contacting the feedstream with a catalyst;
converting the feedstream into a product stream comprising acrylic acid, alkyl acrylate or a cation-balanced acrylate over the catalyst;
and recovering the acrylic acid, alkyl acrylate or a cation-balanced acrylate from the product stream;
where the lactic source comprises lactic acid, an alkyl lactate, a cation-balanced lactate, or combinations thereof.

11. The process of claim 10 wherein the contacting is carried out at a temperature of from about 200° C. to 325° C.

12. The process of claim 10 wherein the catalyst comprises a FAU zeolite.

13. The process of claim 10 wherein the catalyst comprises a LTL zeolite.

14. The process of claim 10 wherein the pressure is greater than 25 barg.

15. The process of claim 10 wherein a selectivity to acrylic acid during dehydration is greater than 50 mol %.

16. The process of claim 15 further comprising contacting the catalyst with a second feedstream comprising an alcohol.

17. The process of claim 16 wherein the alcohol comprises methanol, ethanol, butanol, or mixtures thereof.

18. The process of claim 10 wherein the alkyl lactate comprises methyl lactate, ethyl lactate, butyl lactate, or combinations thereof.

19. The process of claim 10 wherein the feedstream comprises less than 50 wt % water.

20. A process for catalytic dehydration of a lactic source, the process comprising:
pressurizing a feedstream comprising a lactic source to a pressure greater than 25 barg and sufficient to have a liquid phase;
contacting the feedstream with a catalyst comprising a FAU zeolite;
converting the feedstream into a product stream comprising acrylic acid, alkyl acrylate or a cation-balanced acrylate over the catalyst;
and recovering the acrylic acid, alkyl acrylate or a cation-balanced acrylate from the product stream;
where the lactic source comprises lactic acid, an alkyl lactate, a cation-balanced lactate, or combinations thereof.
